(19) United States
(12) Patent Application Publication
(10) Pub. No.: US 2010/0168555 A1
(43) Pub. Date: Jul. 1, 2010

(54) Interventional devices for chronic total occlusion recanalization under MRI guidance

(76) Inventors: Parag V. Karmarkar, Columbia, MD (US); Ergin Atalar, Columbia, MD (US); Robert J. Lederman, Chevy Chase, MD (US); Amish N. Raval, Middleton, WI (US)

Correspondence Address: MCKENNA LONG & ALDRIDGE LLP, 1900 K STREET, NW, WASHINGTON, DC, 20006, US

(21) Appl. No.: 12/654,607

(22) Filed: Dec. 24, 2009

Related U.S. Application Data
(62) Division of application No. 11/131,304, filed on May 18, 2005, now abandoned.
(60) Provisional application No. 60/572,038, filed on May 18, 2004.

(51) Int. Cl. A61B 5/055

(52) U.S. Cl. 600/423

(57) ABSTRACT

Disclosed is a guide catheter that includes one or more RF antennas to enhance the visibility of the guide catheter in MR imagery. One embodiment of the guide catheter includes a loop coil at the distal end of the guide catheter and a loopless antenna between the distal end and the proximal end. By combining a loop coil and a loopless antenna on the catheter, the shaft of the catheter may be visible in MR imagery while the distal end may appear in the MR imagery more brightly than the shaft.

This application is a divisional of U.S. patent application Ser. No. 11/131,304, filed May 18, 2005, and claims the benefit of U.S. Provisional Patent Application No. 60/572,038, filed on May 18, 2004, both of which are hereby incorporated by reference in their entireties for all purposes as if fully set forth herein.

The research and development effort associated with the subject matter of this patent application was supported by the NIH Division of Intramural Research under Z01-HL005062-01 CVB and HL57483.

### BACKGROUND OF THE INVENTION

1. Field of the Invention

The present invention generally relates to catheters, which are introduced into a biological duct, blood vessel, hollow organ, body cavity, or the like, during a medical procedure. More particularly, the present invention relates to catheters that employ one or more RF antennas to improve the visibility of the catheter and the surrounding tissue for various diagnostic and/or therapeutic purposes in an MRI environment.

2. Discussion of the Related Art

Catheters have long been used for the purpose of providing localized therapy by advancing a surgical tool (e.g., a needle, suturing device, stent or angioplasty balloon, delivering drugs, biological materials, etc.) through surrounding anatomy (e.g., the lumen of a blood vessel) to a desired, target area (e.g., a blood vessel occlusion). However, advancement of the catheter requires constant monitoring to ensure that the catheter is advanced through the surrounding anatomy, without kinking, causing injury or failing mechanically. These interventional procedures are often guided by x-ray fluoroscopy imaging.

However, there are a number of limiting characteristics associated with conventional X-ray imaging. X-ray imaging is a 2D projection imaging and cannot identify tortuosity of vasculature. Also, soft tissue visualization by x-ray imaging is not possible. First, conventional X-ray does not provide a full and complete visualization of the vascular geometry. Specifically, X-ray only visualizes a vascular lumen, and only when filled with radiographic contrast. X-ray does not provide an image of the occluded portion of a blood vessel since the contrasting agent injected into the vasculature does not penetrate the occluded segment of the blood vessel. X-ray never visualizes the external (adventitial) border or contour of a vessel. As such, the practitioner does not know the geometry of the occluded portion of the blood vessel. In addition, conventional X-Ray only provides a two dimensional projections. Another limiting feature associated with conventional X-Ray is its inability to provide cross-sectional images of the vasculature. Still another less desirable feature is the exposure of the patient to potentially harmful X-Ray radiation.

Unlike conventional X-Ray, MRI's excellent soft tissue contrast is very capable of providing full and complete images of the vasculature geometry in two or three dimensions, including the outer contour and any occluded portion thereof. Furthermore, MRI can provide multiplaner imaging e.g. axial, sagittal and coronal images, which may enable the accurate guidance of interventional procedures.

Thus excellent soft tissue contrast and multiplaner imaging capability of MRI will enable superior anatomical imaging, however, conventional commercially available interventional devices cannot be visualized in an MRI environment and may not be safe to use in an MRI environment for safety concerns (e.g. RF heating, ferromagnetic issues). Interventional devices may be made visible in an MRI environment by incorporating susceptibility artifacts creating materials in the catheters or by incorporating RF antennas in the catheters. Examples of such devices can be found, for example, in U.S. Pat. No. 5,699,801 and co-pending patent application Ser. No. 10/769,994, the contents of which are incorporated herein by reference. However, there is an ongoing need to further improve the visibility of such devices within the surrounding anatomy to better assist the practitioner.

### SUMMARY OF THE INVENTION

The present invention provides various catheter configurations which incorporate one or more RF antennas to improve the visibility of the catheter and the surrounding anatomy in an MR image. In one configuration, the catheter incorporates one or more loop antennas. In another configuration, the catheter incorporates a loopless antenna. In yet another configuration, the catheter incorporates one or more loop antennas and a loopless antenna. The specific configurations described below provide brighter, more clearly distinguishable signals within the MR image that can be used to better visualize the interventional devices and enable navigating through blood vessels.

Accordingly, one advantage of the present invention is improved MR guidance by providing MR images in which the position of the catheter is more clearly distinguishable in relation to the surrounding anatomy. For example, the present invention provides guide catheters that are visible in MR images along the length of the catheter, and whereby the distal end of the catheter has enhanced visibility in MR images. This is important in vascular procedures such as chronic total occlusion recanalization, in which enhanced visualization helps prevent inadvertent perforation of the blood vessel wall.

Another advantage of the present invention is improved MR guidance by providing MR images in which a distal section of the catheter tip is clearly distinguishable in the surrounding anatomy.

Still another advantage of the present invention is improved MR guidance by providing MR images in which at least a substantial portion of the catheter, including the tip and the shaft of the catheter are clearly distinguishable within the MR image.

In accordance with a first aspect of the present invention, the aforementioned and other advantages are achieved through a guide catheter, which comprises a loop antenna disposed at the distal end of the guide catheter, and a loopless antenna disposed on the guide catheter.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1A illustrates an exemplary guide catheter according to the present invention;

FIG. 1B is a cross sectional view of the guide catheter illustrated in FIG. 1A;

FIG. 1C illustrates an exemplary loop coil guide catheter of the present invention;

FIG. 2A illustrates an exemplary multiple coil guide catheter according to the present invention;

FIG. 2B is a cross sectional view of the multiple coil guide catheter illustrated in FIG. 2A;

FIG. 2C illustrated another exemplary multiple coil guide catheter according to the present invention;

FIG. 3A illustrates an exemplary forward-coiled loopless guide catheter according to the present invention;

FIG. 3B illustrates a rearward-coiled loopless guide catheter according to the present invention;

FIG. 3C is a cross sectional view of the distal end of the rearward-coiled loopless guide catheter illustrated in FIG. 3B;

FIG. 4A illustrates an exemplary hybrid guide catheter according to the present invention;

FIG. 4B illustrated an exemplary hybrid guide catheter employing braided conductors;

FIG. 4C is a cross sectional view of the hybrid guide catheter illustrated in FIG. 4B;

FIG. 4D illustrates an exemplary hybrid guide catheter employing RF chokes;

FIG. 4E is a cross sectional view of the hybrid guide catheter illustrated in FIG. 4D;

FIG. 5 illustrates exemplary RF antenna configurations and corresponding MRI visibility curves;

FIG. 6A illustrates an exemplary multiple coil guidewire according to the present invention;

FIG. 6B is a cross sectional view of the multiple coil guidewire illustrated in FIG. 6A;

FIG. 7 illustrates an exemplary guide catheter with a plurality of susceptibility artifact markers according to the present invention;

FIG. 8 illustrates an exemplary system for acquiring and displaying MR imagery of a guide catheter according to the present invention; and

FIG. 9 illustrates an exemplary display 900 of multiple MRI images according to the present invention.

### DETAILED DESCRIPTION OF THE ILLUSTRATED EMBODIMENTS

The present invention involves the use of an inductor loop coil in conjunction with a guide catheter such that the inductor loop coil (hereinafter “coil”) acts as an antenna that is matched and tuned to the Larmor frequency of MRI (0.25Tesla-11Tesla). This antenna receives RF signal from the surrounding tissue generated in response to external RF energy applied by the MRI system, which the MRI system subsequently detects and displays in MR images.

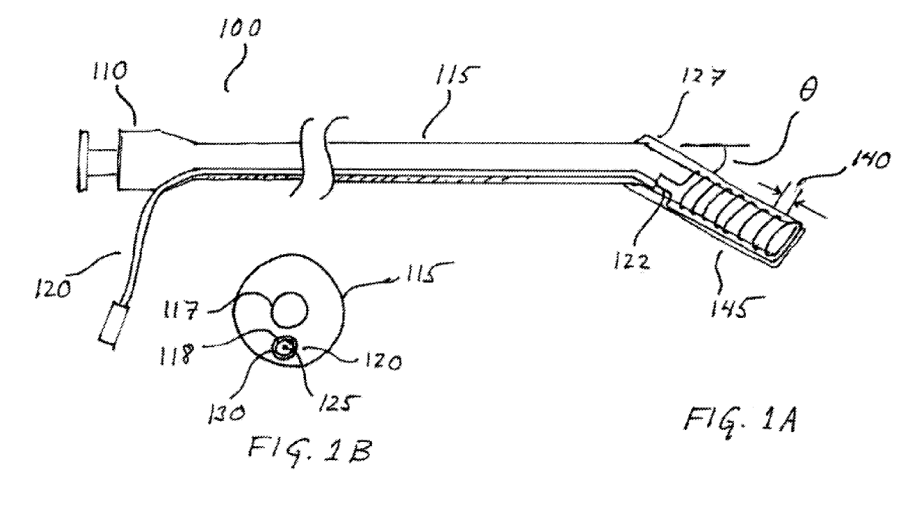

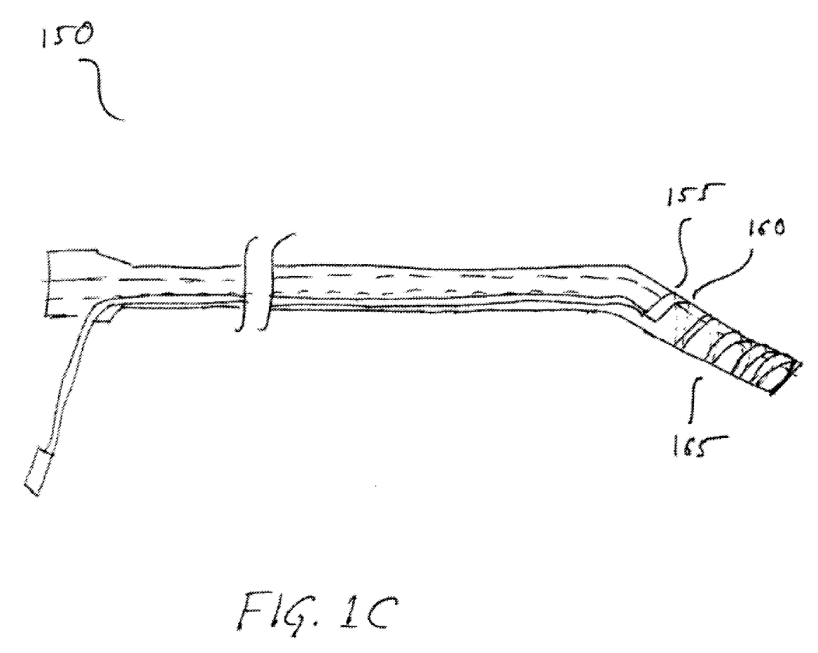

FIG. 1A illustrates an exemplary single loop coil guide catheter 100 according to the present invention. Single loop coil guide catheter 100 includes a multi-lumen polymeric flexible tubing 115, which may be braided, non braided, metallic or non-metallic; a hub 110; a microcoaxial cable 120; and a loop coil 145 formed of a loop wire 122.

As used herein, “microcoaxial cable” refers to a cable having an inner conductor and a shield, wherein the cable has a diameter that makes it suitable for minimally invasive medical use, such as in a catheter.

FIG. 1B is a cross section of guide catheter 100, including multi-lumen polymeric flexible tubing 115 with a central lumen 117 and microlumen 118; and a microcoaxial cable 120 within the microlumen 118, wherein the microcoaxial cable 120 has a shield 130 and an inner conductor 125. The central lumen 117 has a diameter consistent with the diameter of a guidewire or various surgical tools such as a needle or a balloon catheter.

In a particular embodiment, the loop coil has an approximate length of between 0.5-50 cm, and a diameter of about 0.25-15 mm, with a pitch 140 (distance between each turn of the coil) of about 0.05 to 10 mm. The loop wire 122 may be made of a non-magnetic conductive wire, such as copper, gold, gold-platinum, or platinum-iridium. The loop wire 122 should be non-magnetic in order to prevent susceptibility artifacts in acquired MR imagery. One end of the loop wire 125 is connected to the inner conductor 122 of the microcoaxial cable 120, and the other end is connected to the shield 130 of the microcoaxial cable 120.

The loop coil 145 should be formed as close as possible to the distal end of the guide catheter 100, such as within 0.01 mm of the distal end. The loop coil 145 may be wound such that loop wire 122 coils in a direction toward the distal end of catheter 100, or it may coil in a direction toward the proximal end. The loop coil 145 may be coated with a thin polymeric insulation to prevent the loop coil 145 from in contact with body fluids. Although FIG. 1A illustrates a coiled loop 145, other loops may be used, such as a twisted pair loop, a parallel loop, and a coiled loop.

The guide catheter 100 preferably includes a bend having a bend angle θ, which substantially enables an operator to steer the guide catheter 100 within a vascular structure by rotating and steering. The bend angle θ may be between about 20° and about 90°. In a particular embodiment, the bend angle θ is approximately 30°. Alternatively, single loop coil guide catheter 100 may have no such bend, in which case the single loop coil guide catheter 100 may by a deflectable tip catheter, wherein the distal end of the catheter is capable of deflection in one or more directions.

FIG. 1C illustrates an exemplary guide catheter 150, in which coils may be made whereby the positive wires 155 and the ground wires 160 run parallel to each other along the length of the coiled section 165.

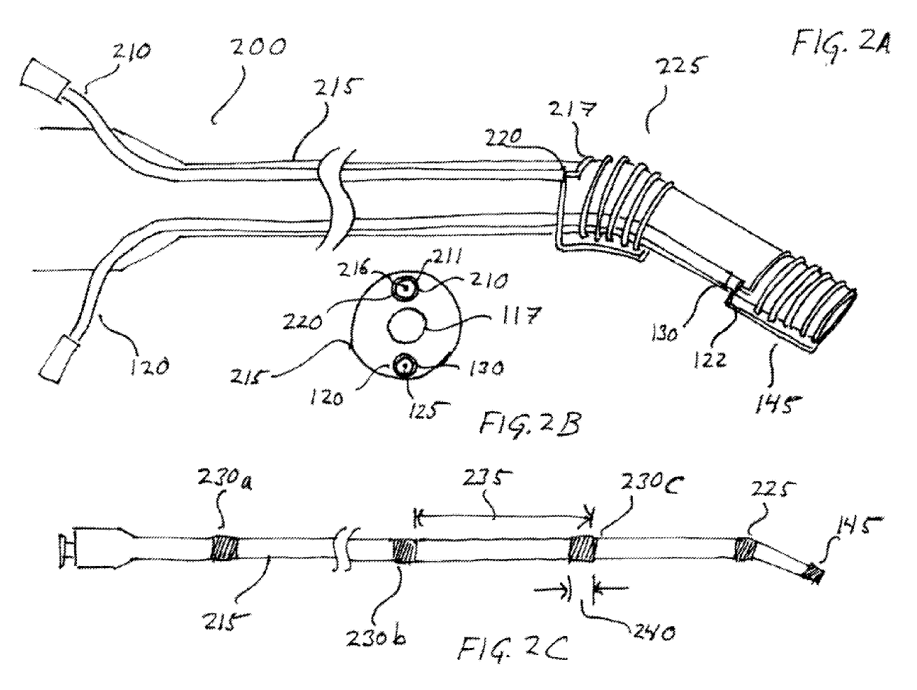

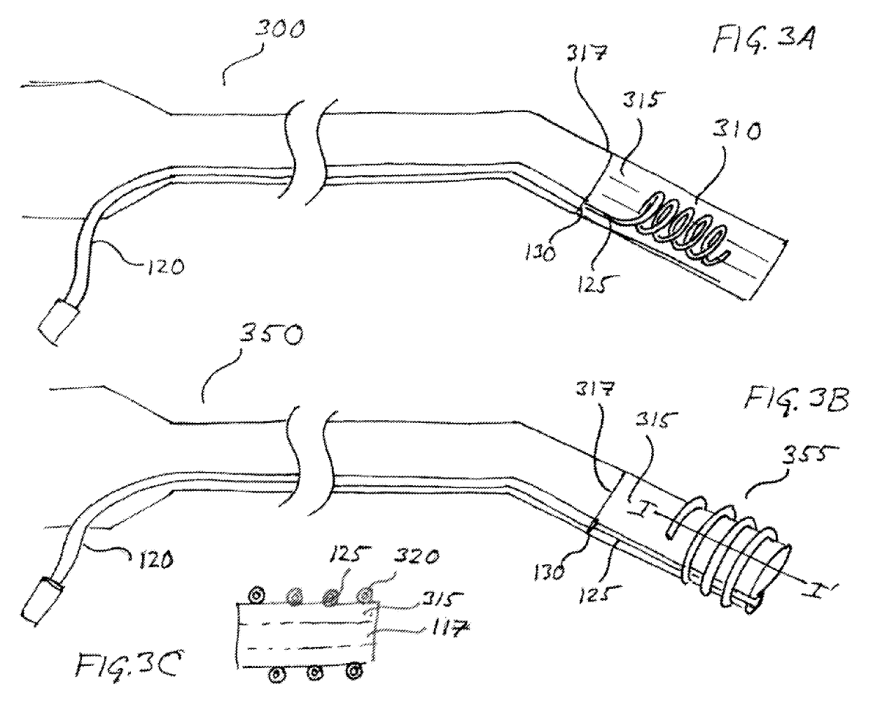

FIG. 2A illustrates an exemplary multiple coil guide catheter 200 according to the present invention. The configuration of multiple coil guide catheter 200 may be similar to guide catheter 100, with the addition of a second microcoaxial cable 210 and a second loop coil 225.

FIG. 2B is a cross sectional view of exemplary multiple coil guide catheter 200. As illustrated, guide catheter 200 includes a flexible tubing 215; a central lumen 117; a microlumen 118; a microcoaxial cable 120, which has a shield 130 and an inner conductor 125; a second microlumen 211; and a second microcoaxial cable 210, which includes an inner conductor 216 and a shield 220.

As stated, multiple coil guide catheter 200 includes a second loop coil 225, which is formed of a second coil wire 217. One end of second coil wire 217 is connected to the inner conductor 216 of the second coaxial cable 210, and the other end is connected to the shield 220 of microcoaxial cable 210. Loop coils 145 and 225 may be in close proximity to each other and separated by a distance of 1 mm or more.

FIG. 2C illustrates an exemplary embodiment of multiple coil guide catheter 200, which includes multiple loop coils 145, 225, and 230a-c. Loop coils 230a-c may have characteristics different from those of loop coils 145 and 225 so that they are distinguishable from the latter loop coils in MR imagery. The loop coils 230a-c may be spaced such that loop coil 230c may be anywhere from 1-10 cm from second coil 225. Loop coils 230a-230c may have a length 240 between 2 mm and 1 cm, depending on the diameter of guide catheter 200. The spacing 235 between loop coils 230a-230c depends on the clinical use for the guide catheter 200. In a particular embodiment, spacing 235 is about 0.5-1 cm.

In a particular embodiment, length 240 is approximately equal to the diameter of the guide catheter shaft (or the diameter of the coil 230a, 230b, or 230c) so that each coil 230a-c may appear as a “square” feature in MR imagery. Thus, image processing software can more easily determine the centroid corresponding to each of loop coils 230a-c. Loop coils 230a-c may be evenly spaced from each other by distance 235. This in turn makes it easier for the image processing software to determine the distances between the centroids of each of the coils and compare them with the known distance 235. This may be useful for various reasons. For example, if the image processing software determines that two centroids are considerably closer together than known distance 235, it may be because the guide catheter 200 is buckling or is kinked.

Loop coils 230a-c may have as tight a pitch as possible in order to maximize RF flux impinging on each of the coils by having as many turns as possible within length 240.

In the exemplary embodiment illustrated in FIG. 2C, the multi-lumen polymeric flexible tubing 215 may have one microlumen for each of the coils 230a-c, the loop coil 145, and the second loop coil 225.

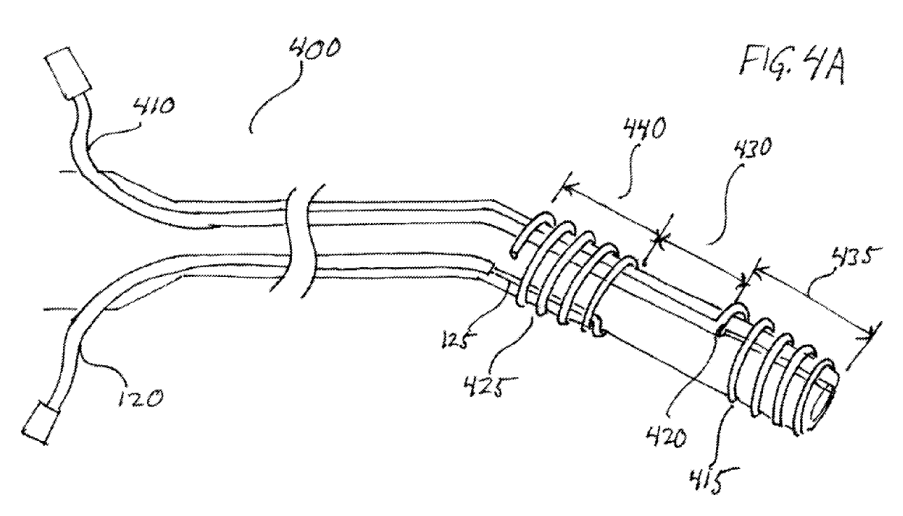

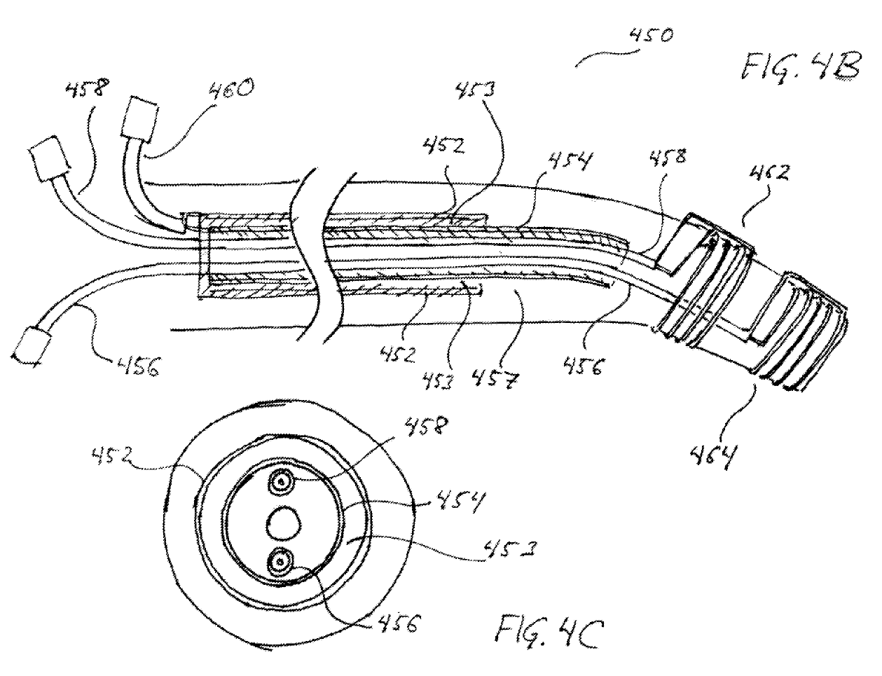

FIGS. 3A and 3B illustrate exemplary guide catheters, which employ loopless antennas. FIG. 3A illustrates an exemplary forward-coiled loopless guide catheter 300, which includes a microcoaxial cable 120, and a coil 310, which terminates without forming a loop. The shield 130 of the microcoaxial cable 120 terminates approximately 0.5-1 cm from the distal end of the guide catheter 300. Inner conductor 125 extends in the direction of the distal end of guide catheter 300 to form a coil 310. The coil 310 may be embedded within a thick insulating material 315, which extends beyond where the flexible polymeric tubing ends at interface 317. The inner conductor 125 may be covered in a thin polymeric coating for the length beyond the termination of the shield 130. The inner conductor 125 may have a straight and coiled portion beyond the termination of the shield 130. For example, the inner conductor 125 may have a straight portion of length of about 1-30 cm beyond the termination of the shield 130, and a coil 310 about 0.2-10 cm long.

FIG. 3B illustrates a rearward-coiled loopless guide catheter 350, which is substantially similar to guide catheter 300, except that the inner conductor 125 of the microcoaxial cable 120 remains substantially straight until it reaches the distal end of the guide catheter 350, and then coils rearward, toward the proximal end. In this exemplary embodiment, the inner conductor 125, which is sheathed in a thin polymeric tubing 320, is wrapped around the outside of the thick insulating material 315. The inner conductor 125 may exit the thick insulating material 315 at the distal tip of the guide catheter 350 and then coil around the outside of the thick insulating material for a distance of about 0.2-1 cm. As with loopless guide catheter 300, inner conductor 125 may have a straight portion of length of about 1-30 cm beyond the termination of the shield 130.

FIG. 3C is a cross sectional view of the distal end of guide catheter 350, as taken along cross sectional line I-I′. FIG. 3C illustrates thick insulating material 315, which continues the central lumen 117; inner conductor 125; and thin polymeric tubing 320.

In an alternate embodiment, the inner conductor 125 may be substantially straight. In this case, the inner conductor may be similar to a standard dipole.

The loopless antennas described above may be formed of an inner conductor 125 of a microcoaxial cables, or may be formed of separate nonmagnetic conducting material that is connected to the inner conductor 125.

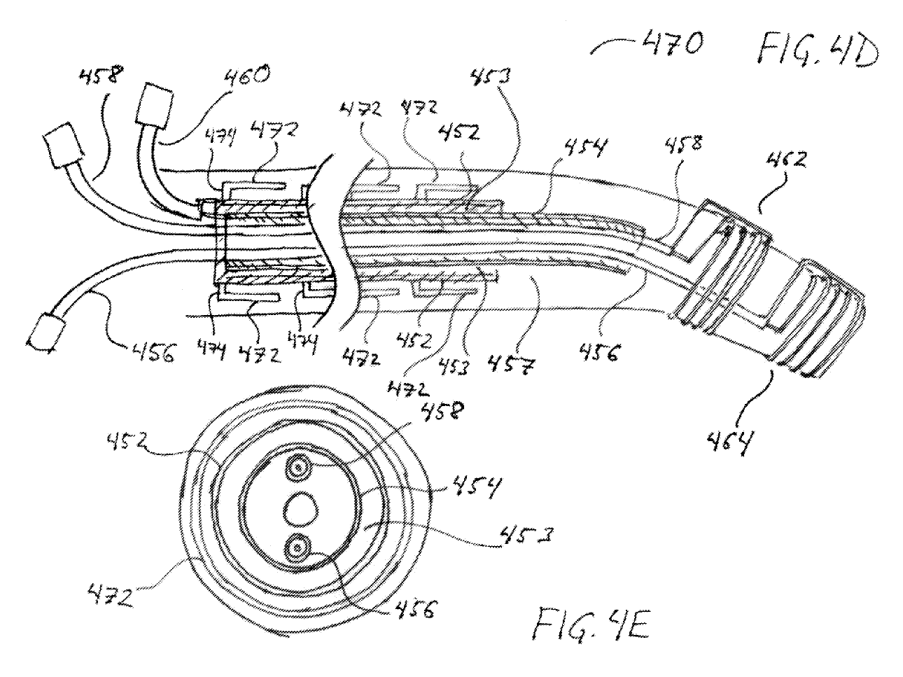

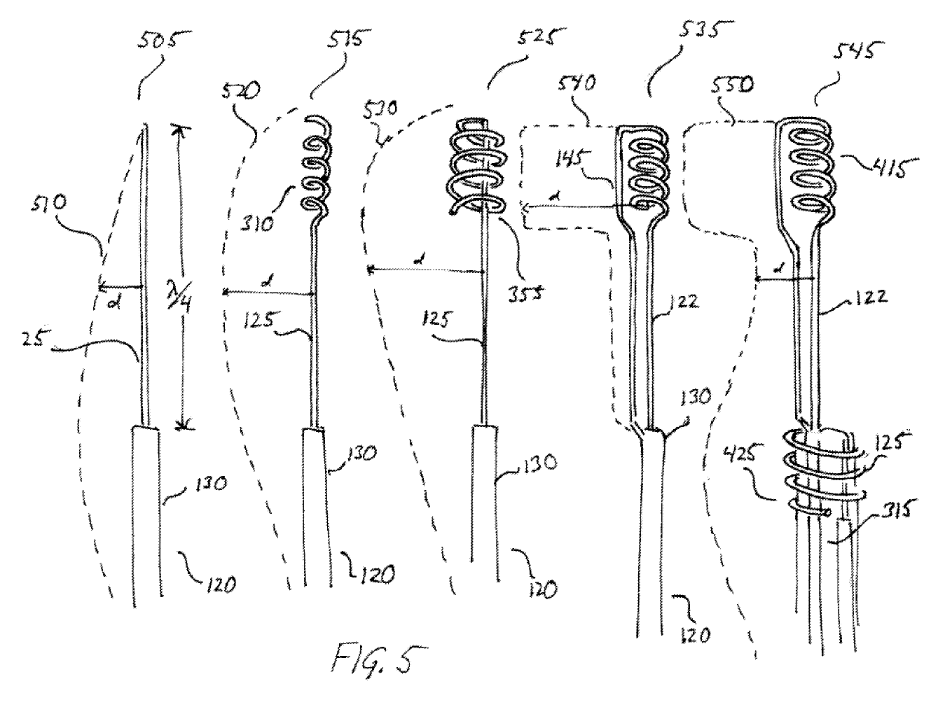

FIG. 4A illustrates an exemplary hybrid guide catheter 400 according to the present invention. The hybrid guide catheter 400 includes a loop coil 415 and a loopless coil 425. The loop coil 415 may be substantially similar to the loop coil 145 of the single loop coil guide catheter 100, and the loopless coil may be substantially similar to either the loopless coil 355 of the rearward-coiled loopless catheter 350, or the loopless coil 310 of the forward-coiled loopless catheter 300. The two coils may separated by a distance of about 3 cm to about 5 cm to prevent RF coupling between them. Alternatively, the positive conductor of the loopless coil 355 may instead be substantially straight.

FIGS. 4B and 4C illustrate another hybrid guide catheter 450 according to the present invention. Hybrid guide catheter 450 has a loopless antenna that may be build into the walls of the guide catheter 450. This Hybrid guide catheter 450 includes an outer shield braid 452; and inner braid 454 substantially concentric to and extending beyond the outer shield braid 452; and an insulator 453 disposed between the outer shield braid 452 and the inner braid 454. The hybrid guide catheter 450 further includes a microcoaxial cable 460, wherein the microcoaxial cable 460 has an inner conductor connected to the inner braid 454 and a shield connected to the outer shield braid 452 at the proximal end of the guide catheter 450. The hybrid guide catheter 450 also includes a loop coil 462 with one end connected to inner conductor microcoaxial cable 458 and the other end connected to the shield of microcoaxial cable 458, which may be connected to ground. The hybrid guide catheter 450 further includes another loop coil 464 with one end connected to the inner conductor of microcoaxial cable 456 and the other end connected to the shield of microcoaxial cable 456.

The microcoaxial cables 456, 458, and 460 are connected at the proximal end to matching tuning circuitry which matches and tunes the output of the antennas to the Larmor frequency (used in MRI) and decouples the output of the antennas during RF transmit by the MRI scanner.

For purposes of illustration, hybrid guide catheter 450 has two loop coils 462 and 464. It will be readily apparent to one of ordinary skill that one loop coil or multiple loops coils are possible and within the scope of the invention.

In hybrid guide catheter 450, the inner braid 454 and the outer shield braid 452 form a loopless antenna 457, in which the inner braid 454 serves as the positive conductor of the loopless antenna, and the outer shield braid 452 serves as a shield.

The mechanical characteristics of the inner braid 454 and the outer shield braid 452 offers the advantage of efficiently transferring torque from the proximal end to the distal end of hybrid guide catheter 450, and substantially evenly distributing axial forces along its length (i.e., “pushability”). These mechanical characteristics are desirable in any guide catheter in that they affect an operator's ability to steer the distal end of the hybrid guide catheter 450 during procedures in which precise steering of the guide catheter 450 is required, such as in chronic total occlusion recanalization and other vascular interventions. In chronic total occlusion recanalization, precise steering of a guide catheter is required to, among other things, prevent inadvertent perforation of a blood vessel wall.

FIGS. 4D and 4E illustrate a hybrid guide catheter 470 is substantially similar to hybrid guide catheter 450 illustrated in FIGS. 4B and 4C, except that hybrid guide catheter 470 includes a plurality of RF chokes 472. RF chokes 472 may comprise a concentric braid, as illustrated in FIG. 4E, which is divided into segments along an axis defined by the concentric axis of the guide catheter 470. Each segment is connected to the outer shield braid 452 by connection part 474, which may be an extension of the braid forming RF choke 472. Alternatively, connection part 474 may include wires that connect the braid within RF choke 472 to the outer shield braid 452.

The presence of RF chokes 472 prevents an RF standing wave from occurring along the guide catheter 470, which may cause RF-induced heating of the guide catheter 470. This, in turn, could pose a safety hazard for the patient. This is particularly important for long guide catheters, for example, guide catheters that are longer than 50 cm. Accordingly, RF chokes 472 may enhance the safety of the guide catheter 470 by substantially preventing RF heating of the catheter in an MRI environment.

FIG. 5 illustrates five exemplary RF antenna configurations for guide catheters, and representative MRI visibility curves corresponding to each RF antenna configuration. The MRI visibility curves represent the sensitivity of a given RF antenna configuration. The horizontal distance d from an axis defined by inner conductor 125 refers to the sensitivity of the antenna at that particular point along the axis. Each MRI visibility curve is the locus of the sensitivities illustrated by distance d for each point along the axis defined by the inner conductor 125.

RF antenna configuration 505 includes a straight loopless antenna, which is described above. The inner conductor 125 of the microcoaxial cable 120 extends beyond the shield 130 of the microcoaxial cable 120, preferably by a distance of λ/4, where λ is the RF wavelength to be received by the RF antenna configuration 505. The MRI visibility curve, and thus the sensitivity of the antenna, corresponds to a current density induced within the inner conductor 125 in response to RF energy of wavelength λ impinging on the inner conductor 125. Since the loopless antenna is not an inductor loop, there is no net current flow; therefore the current density (and thus the MRI visibility) is substantially zero at the distal end of the inner conductor 125, as illustrated.

MRI visibility curve 510 may represent the sensitivity of loopless antenna 457 formed by the inner braid 454 and the outer shield braid 452 of hybrid guide catheters 450 and 470.

RF antenna configuration 515 corresponds to the forward-coiled loopless guide catheter 300, which is described above and illustrated in FIG. 3A. RF antenna configuration 520 is loopless with a coil shape at the distal end, which inductively captures a greater amount of RF flux at the distal end than does RF configuration 505. In RF antenna configuration 515, the diameter of the coil, and the increased length of inner conductor 125 present in the coil 310 (in contrast to the straight loopless antenna) in the proximity of the distal end results in a greater current density in the proximity of the distal end. Accordingly, the MRI visibility curve 520 indicates increased visibility (due to increased sensitivity, which is due to increased current density) near the distal end of RF antenna configuration 515.

RF antenna configuration 525 corresponds to the rearward-coiled loopless guide catheter 350 illustrated in FIG. 3B. This configuration has a similar MRI visibility curve to MRI visibility curve 520. However, the MRI visibility curve 530 indicates even greater sensitivity in the vicinity of the distal end. This is due to the fact that the coil 355 of guide catheter 350 has a greater diameter because it wraps around the outside of thick insulating material 315 as illustrated in FIG. 3B, and thus coil 355 receives more RF flux. Coil 310 of guide catheter 300 is embedded within thick insulating material 315, as illustrated in FIG. 3A, and thus has a smaller diameter. Accordingly, RF antenna configuration 525 has greater MRI visibility, as illustrated by the MM visibility curve 520, than does RF antenna configuration 515.

RF antenna configuration 535 corresponds to single loop coil guide catheter 100 illustrated in FIG. 1A. RF antenna configuration 535 has a loop wire 122, which completes a circuit between the inner conductor 125 and the shield 130 of microcoaxial cable 120. RF antenna configuration 535 has a strong sensitivity, which corresponds to MRI visibility curve 540. The primary sensitivity of RF antenna configuration 535 is in the radial direction, outward from an axis defined by the loop coil 145. Accordingly. RF antenna configuration 535 provides for very strong MRI visibility in the vicinity of the distal end, as illustrated by the MRI visibility curve 540, but significantly less MRI visibility everywhere else.

RF antenna configuration 545 corresponds to hybrid guide catheter 400 illustrated in FIG. 4A, which may comprise the combination of coiled loopless antenna 525 and single loop coiled antenna 535, wherein the coiled loopless antenna 525 is translated “downward” relative to the single loop antenna 535. Due to superposition, the MRI visibility curve 550 corresponding to RF antenna configuration 545 indicates good MRI visibility along the length of the catheter and greater MRI visibility in the vicinity of the distal end. As such, the RF antenna configuration of hybrid guide catheters 400, 450 and 470 provides for a greater overall MRI visibility, whereby the entire catheter is visible in MR imagery, and the distal end has enhanced visibility. This MRI visibility feature may be extremely useful in certain medical procedures, such as chronic total occlusion recanalization, wherein the operator needs to very clearly see the distal end of the guide catheter relative to the surrounding tissue in order to prevent inadvertent perforation of a blood vessel wall, and wherein the operator needs to see the entire length of the guide catheter to prevent buckling and kinking of the guide catheter.

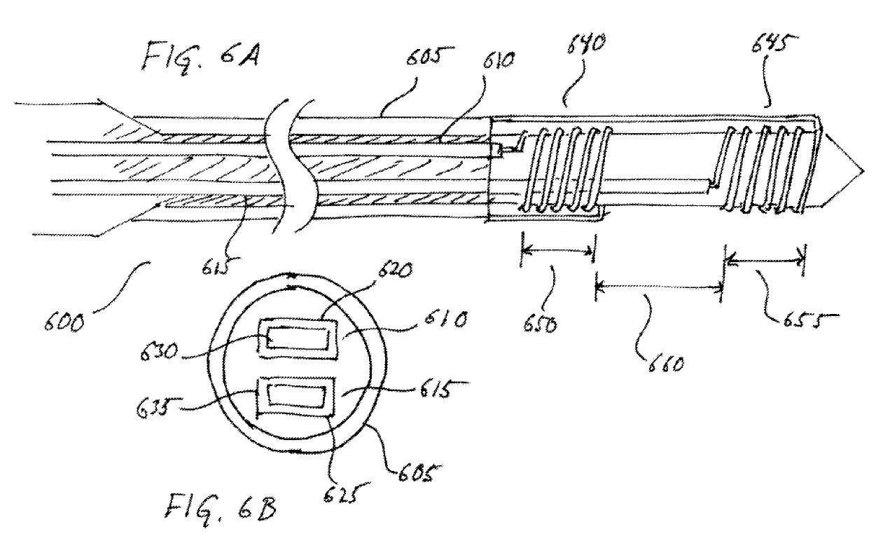

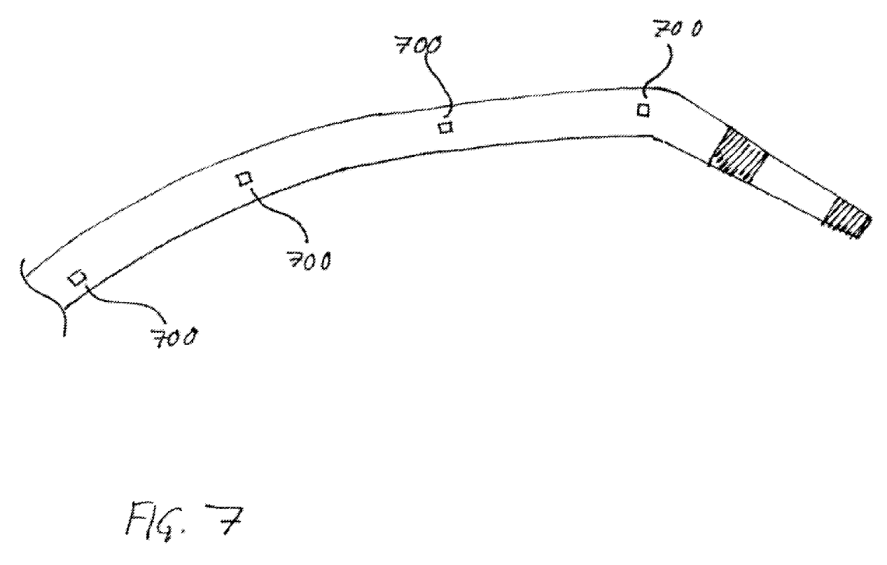

FIGS. 6A and 6B illustrate an exemplary multiple coil guidewire 600 according to the present invention. The guidewire 600 includes a shield 605; two inner insulated conductors 610 and 615; and two loop coils 640 and 645, which are respectively connected to inner insulated conductors 610 and 615.

The guidewire 600 may have an overall length of about 120 cm, with 40 cm of that distance constituting the distal section of the guidewire 600. The distal section of the guidewire 600 may be made flexible by heat treating it at 450° C. for 90 minutes. The shield 605 may be made of Nitinol, although other non-ferrous flexible conductive materials may be used that have mechanical characteristics, such as the ability to efficiently transfer torque and equally distribute and transfer axial torque (i.e., “pushability”). The shield 605 may be in the form of a tube or a closely wound coil. Further, the distal section may also be a closely wound wire instead of a tubing. The insulator 620 and 625 disposed on inner conductors 620 and 625 may include FEP (fluorinated ethylene propylene).

Loop coil 640 is formed of inner conductor 610, which is connected to the shield 605 at the other end of its loop. Loop coil 645 is formed of inner conductor 615, which connects to the shield 605 at the other end of its loop. Both inner conductors 610 and 615 may include materials such as pt-ir, gold-ir, and MP35N. Loop coils 640 and 645 may each have a length between about 0.2-10 cm between In a particular embodiment, loop coils 640 and 645 respectively have a length 650 and 655 of less than about 0.5 cm and are spaced apart by a distance 660 about 0.5 cm, although distance 660 may be as high as 1 cm.

Although guidewire 600, as illustrated in FIG. 6A, has two loop coils, coil 640 may be a loopless coil, as described above. Whether coil 640 is a loop coil or a loopless coil depends on how an operator wishes the guidewire to appear in MR imagery. For example, for the guidewire 600 illustrated in FIG. 6A, coils 640 and 645 may be the only visible components of the wire. This is because the inner conductors 610 and 615 are connected to the shield 605. In this case only the portion of inner conductors 610 and 615 exposed from the shield (i.e., the coils 640 and 645) behave as RF antennas.

If coil 640 is configured as a loopless coil, inner conductor 610 terminates without being connected to shield 605. In this case, both the coil 640 and the inner conductor 610 will behave as an RF antenna, which may be represented by MRI visibility curve 530 illustrated in FIG. 5. Further, combining the loopless coil 640 and loop coil 645 on the guidewire 600 as illustrated in FIG. 6A may result in an RF antenna, which may be represented by MRI visibility curve 550 illustrated in FIG. 5. In this case, the operator may see substantially the entire guidewire 600, with the distal end of the guidewire 600 appearing brighter than the rest of the guidewire 600 in the MR imagery. This is important for procedures such as chronic total occlusion recanalization, whereby the operator needs to clearly see distal end of the guidewire to prevent inadvertent perforation of a blood vessel wall, and whereby the operator needs to see substantially the entire length of the guide catheter to prevent buckling and kinking.

Guidewire 600 may employ braids and RF chokes in a manner substantially similar to guide catheters 360 and 370 respectively illustrated in FIGS. 4D and 4E. All the loop and loopless RF antennas of the guidewire are matched and tuned to the Larmor frequency by external circuitry. The external circuitry may also include a decoupling circuit, which detunes the coil during RF transmit by the MRI scanner. This circuitry may be incorporated on the guide catheter or may be housed separately outside the catheter. Each individual coil typically has a separate circuit.

Any of the above configuration of guidewire 600 may be used with any of the guide catheters described above. However, the configuration of guidewire 600 with the loop coil 640 may be preferable in that it may be less prone to RF coupling with the coils on the guide catheter.

FIG. 7 illustrates a guide catheter according to the present invention with one or more susceptibility artifact markers 700 disposed on or within the tubing of the guide catheter. The susceptibility artifact markers 700 have magnetic properties that distort the MRI magnetic field in their immediate vicinity and thereby intentionally create an anomaly in the MR imagery at its location. The susceptibility artifact markers 700 may include paramagnetic materials such as dysproxium oxide, iron, steel, and nickel.

Accordingly, the susceptibility artifact markers 700 may serve as passive fiducial markers whereby the position and curvature of the guide catheter may be determined in the MR imagery. These markers may supplement the coils described above in providing MR imagery of the guide catheter. Further, the passive nature of the susceptibility artifact markers 700 may provide as a reliable “backup” for identifying the guide catheter in MR imagery in the event of coil failure, for example, a break in a microcoaxial cable or a failure in an impedance matching circuit.

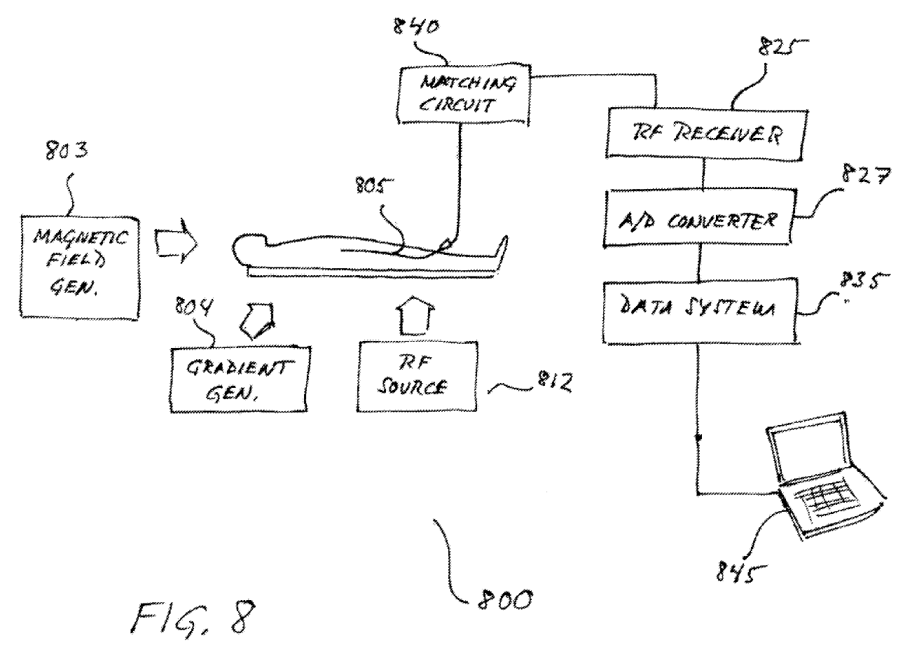

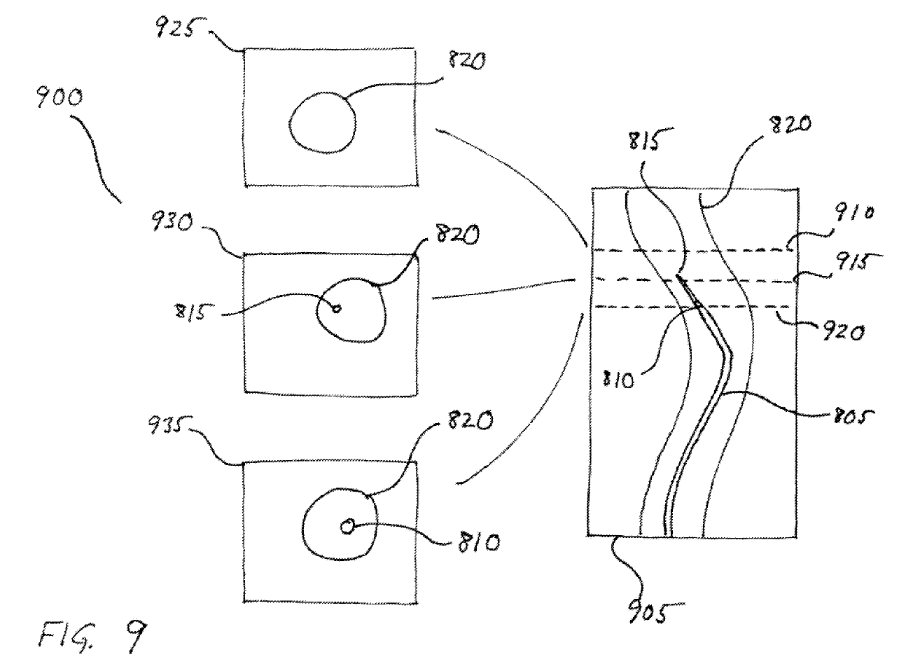

FIG. 8 illustrates an exemplary system 800 for acquiring and displaying MR imagery of an exemplary guide catheter 805, guidewire, and surrounding anatomy, according to the present invention. System 800 includes a magnetic field generator 803; a gradient generator 804; an RF source 812; and RF receiver 825; an A/D converter 827; a data system 835 with a computer readable medium encoded with software (hereinafter the “software”) for processing and displaying MR imagery; a user interface 845; a guide catheter 805, which may include a guidewire; and a matching circuit 840 connected to the guide catheter 805 and guidewire.

Guide catheter 805 may be any one of the exemplary guide catheters described above. Each coil in the guide catheter 805 may be connected to a corresponding matching circuit 840. The matching circuit 840 matches and tunes the output of the coils on the guide catheter and the guidewire to the Larmor frequency (used in MRI). The matching circuit also includes a decoupling circuit, which detunes each coil during RF transmit by the RF source 812. The matching circuit may be incorporated on the guide catheter 805 or may be housed separately. The matching circuit includes a separate circuit for each individual coil in guide catheter 805.

The data system 835 may include one or more computers that may operate remotely over a network. The software may be stored and executed on the data system 835 or may be stored and executed in a distributed manner between the data system 835 and the user interface 845.

The user interface 845 may include a workstation that is connected directly to the data system 835 or may include computers that are remotely located and connected over a network. It will be apparent to one skilled in the art that many data system and user interface configurations are possible and within the scope of the invention.

FIG. 9 illustrates an exemplary display 900 of multiple MRI images, which may be processed and displayed by the software. The software may display a main image 905, which may be taken along the sagittal plane, the coronal plane, or some vector combination of the two. The software may also display a plurality of cross section images 925, 930, and 935, each of which correspond to a different axial plane. For example, cross sectional image 935 may correspond to axial plane 920; cross sectional image 930 may correspond to axial plane 915; and cross sectional image 925 may correspond to axial plane 910.

The blood vessel 820 may be visible in each image, as illustrated in FIG. 9. As the guide catheter 805 and guidewire are inserted through blood vessel 820, the guidewire tip 815 may be visible in cross sectional image 820 once the guidewire tip 815 enters axial plane 915. Guide catheter distal end 810 may be visible in cross sectional image 935 when the distal end 810 enters axial plane 920. Cross sectional image 925, which corresponds to axial plane 910, represents where the guidewire tip 815 will subsequently appear as the guide catheter 805 or the guidewire is further inserted. Accordingly, cross sectional images 935, 930, and 925 may provide feedback to an operator regarding where the guide catheter distal end 810 is, where the guidewire tip 815 is, and where the guidewire tip 815 will be.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A guide catheter having a distal end and a proximal end, the guide catheter comprising:
a flexible tubing;
an outer conductive braid within the flexible tubing, the outer conductive braid being substantially concentric to the flexible tubing;
an inner conductive braid within the outer conductive braid, wherein the inner conductive braid and the outer conductive braid form a loopless antenna; and
a loop antenna disposed at the distal end of the guide catheter.

2. The guide catheter of claim 1, further comprising an insulator between the outer conductive braid and the inner conductive braid.

3. The guide catheter of claim 1, further comprising a microcoaxial cable disposed within the flexible tubing, wherein the loop antenna is connected to the microcoaxial cable.

4. The guide catheter of claim 1, further comprising a second loop antenna disposed between the first loop antenna and the loopless antenna.

5. The guide catheter of claim 1, further comprising a plurality of RF chokes disposed on a surface of the outer conductive braid.

6. The guide catheter of claim 1, further comprising a microcoaxial cable having an inner conductor connected to the inner conductive braid and having a shield connected to the outer conductive braid.

7. A guide catheter having a distal and proximal end, the guide catheter comprising:
a flexible tubing;
a first microcoaxial cable disposed within the flexible tubing;
a second microcoaxial cable disposed within the flexible tubing;
a loop antenna disposed at the distal end of the guide catheter, wherein the loop antenna is connected to the first microcoaxial cable; and
a loopless antenna disposed between the loops antenna and the proximal end of the guide catheter, wherein the loopless antenna is connected to the second microcoaxial cable.

8. The guide catheter of claim 7, wherein the loop antenna comprises a coil.

9. The guide catheter of claim 7, wherein the loopless antenna comprises a coil that is disposed on the flexible tubing in the direction of the proximal end.

10. The guide catheter of claim 7, wherein the loopless antenna comprises a coil that is disposed on the flexible tubing in the direction of the distal end.

11. The guide catheter of claim 7, wherein the loop antenna and the loopless antenna are separated by about 3 cm to about 5 cm.

12. The guide catheter of claim 7, further comprising a plurality of susceptibility artifact markers disposed on the flexible tubing.

13. A guidewire for use in conjunction with a catheter, the guidewire comprising:
a guidewire microcoaxial cable having a shield;
a first loop antenna disposed at a distal end of the guidewire; and
a second loop antenna,
wherein the first loop antenna and the second loop antenna are connected to the shield.

14. The guidewire of claim 13, wherein the first loop antenna comprises a loop having a length of less than 10 cm.

15. The guidewire of claim 13, wherein the second loop antenna comprises a loop having a length of less than 10 cm.

16. The guidewire of claim 13, wherein the first loop antenna and the second loop antenna are spaced apart by a distance substantially equal to 0.5 cm to 1 cm.

17. The guidewire of claim 13, wherein the shield of the guidewire microcoaxial cable includes Nitinol.

18. A guide catheter for chronic total occlusion recanalization, the guide catheter having a distal end and a proximal end, the guide catheter comprising:
a flexible tubing;
an outer conductive braid within the flexible tubing, the outer conductive braid being substantially concentric to the flexible tubing;
an inner conductive braid, substantially concentric to the outer conductive braid, the inner conductive braid extending beyond a termination of the outer conductive braid and toward the distal end;
an insulator between the outer conductive braid and the inner conductive braid;
a microcoaxial cable disposed within the flexible tubing, the microcoaxial cable having an inner conductor and a shield; and
a loop antenna disposed at the distal end of the guide catheter, wherein the loop antenna is connected to the microcoaxial cable.

19. The guide catheter of claim 18, further comprising a plurality of RF chokes disposed on a surface of the outer conductive braid.

20. The guide catheter of claim 18, further comprising a second loop antenna.
